(19) United States
(12) Patent Application Publication
(10) Pub. No.: US 2006/0200121 A1
(43) Pub. Date: Sep. 7, 2006

(54) Navigable, multi-positional and variable tissue ablation apparatus and methods

(76) Inventor: Thomas M. Mowery, El Macero, CA (US)

Correspondence Address: LAW OFFICE OF KENNETH A. MURRAY, JR., P.O. BOX 2188, DAVIS, CA, 95617, US

(21) Appl. No.: 11/367,135

(22) Filed: Mar. 3, 2006

Related U.S. Application Data
(60) Provisional application No. 60/658,112, filed on Mar. 3, 2005.

(51) Int. Cl. A61B 18/14

(52) U.S. Cl. 606/41

(57) ABSTRACT

A spinal nerve tissue ablation apparatus includes a stylet needle with a distal end having a rounded blunt tip sufficiently sharp to penetrate tissue but sufficiently blunt to avoid impinging on bony surfaces of spinal vertebra. The apparatus includes an energy delivery device having at least a first electrode and a second electrode. Each electrode has a tissue piercing distal end and is positionable in the stylet as the stylet is advanced through tissue. The first and second electrodes are deployable with curvature from the stylet. The stylet includes rotational means for orienting the first and second electrodes and directing the extension according to the curvature of the electrodes. The stylet further includes an optical imaging module to provide continual progressive feedback of ablation surface development. The apparatus further includes an ultra-wide band radio frequency scanning device capable of accurately determining the location of the electrodes within the spinal structure.

### CROSS REFERENCE TO RELATED APPLICATIONS

Priority is claimed to U.S. Provisional Application No. 60/658,112 filed Aug. 10, 2004, titled “NAVIGABLE, MULTI-POSITIONAL AND VARIABLE TISSUE ABLATION APPARATUS AND METHODS” which is referred to and incorporated herein in its entirety by this reference.

### FEDERALLY-SPONSORED RESEARCH

Not Applicable

### SEQUENCE LISTING OR PROGRAM

Not Applicable

### DESCRIPTION

### BACKGROUND OF THE INVENTION

1. Field of the Invention

This invention relates generally to a tissue ablation apparatus and methods for ablating tissue using radiofrequency energy, and more particularly, to such an apparatus and methods capable of effectively navigating the spinal structure and using variable width, length and orientation of electrode tines to allow effective and controlled, multi-positional creation of a desired shape lesion or treatment zone.

2. Description of the Related Art

Historically, numerous techniques have been used to selectively destroy nervous tissue in the brain, spine and elsewhere in the body. The most important of these techniques include: cryogenic surgery, focused ultrasound, chemical destruction, ionizing radiation, mechanical methods, lasers, radiofrequency heating, and direct current heating. Of these methods, the radiofrequency (RF) and direct current heating methods involve the passage of current from an electrode to the surrounding tissue, thereby heating and destroying the tissue with some volume in the vicinity of the electrode. In the peripheral nervous system, RF lesion-making is beginning to make possible the accurate and highly selective destruction of pain-carrying nerve fibers. Temperature is recognized as the fundamental lesion parameter.

Since original development in the early 1900's, radiofrequency treatment of tissue has evolved in medicine to become one of the most effective and widely used techniques for selective destructive and non-destructive treatment of tissue. Generally, insulated electrode needles with a variable amount of non-insulated tip are placed either adjacent or into a target tissue. The electrode needles, connected to an energy generation device, deliver high frequency waves above 250 kilocycles to the target tissues. Typical frequencies are in the range between 500 kHz and 1000 kHz. The energy flows from the electrodes through the target tissue; resistance to the energy flow creates heat within the target tissue. Typically, tissue temperatures above 42 degrees Centigrade will cause destruction to nerve tissue; tissue temperatures below 42 degrees Centigrade will not cause tissue destruction. Pulsed applications of energy are used to maintain tissue temperature below a target level.

The energy delivery device can operate in either a monopolar or bipolar manner. In monopolar operation, energy flows to a dispersing grounding pad located on the exterior of the individual being treated. In bipolar operation, energy flows to an adjacent needle creating a controlled delivery of heat to the tissue within the target area or field of the two needles. The RF current heats the tissue, and the tissue heats the electrode tip, allowing accurate monitoring of tissue temperature. However, measuring tissue temperature is still an indirect method of determining the level of tissue ablation and it is believed that it would be very beneficial to have other means by which to directly determine the progress of the creation of a lesion, for example, using optical imaging techniques. Additionally, current temperature sensing methodologies are unable to account for the heat which may develop in surrounding tissues, e.g., bone, creating an additional thermal reservoir of heat, which will affect time constants and complicate treatment procedures.

Research indicates that electrode tips of about 1.1 mm in diameter and 3 to 5 mm in length produce lesions of about 3 mm circumference or diameter and 4 to 7 mm in length when temperatures are at 65 to 75 degrees Centigrade. Other research indicates that very fine gauge electrodes of 0.25 mm (31 gauge) in diameter with 2 mm long tips give rise to lesion sizes on the order of between 0.7 mm to 0.9 mm in diameter and 1.8 to 2.2 mm at 75 degrees Centigrade for 15 seconds.

Radiofrequency lesioning technology has been used in many medical therapy applications including heart rhythm control, snoring abatement, cancer tumor ablation and pain management. Within the field of pain management, radiofrequency ablation has been used to create lesions in spinal cords, dorsal root ganglia, sympathetic nerves, small nerves to both large and small joints, such as the sacroiliac and facet joints, neuromas, feet, and, vertebral column discs. The lesions created in the target nerve tissues interrupt pain signals from the targeted areas, frequently providing relief from chronic pain.

Traditionally, spinal radiofrequency ablation has been applied in a monopolar manner using single, non-insulated needle tips from 4 to 10 millimeters long. Introducer needles of 18, 20 and 22 gauge are typically used for delivery of the electrode to a target site. For clarification, an 18 gauge needle has an outer diameter of 1.27 mm; a 20 gauge needle has an outer diameter of 0.902 mm; a 22 gauge needle has an outer diameter of 0.711 mm; a 25 gauge needle has an outer diameter of 0.508 mm. The 25 gauge needle is typically not used for electrode delivery because it tends to be too flimsy to effectively penetrate percutaneous tissue.

A single needle ablation creates fairly discrete lesions designed to interrupt the flow of pain signals along the targeted nerve tissue. However, a physician has great difficulty in attempting to place a single needle electrode tip adjacent to target nerves due to several factors including: (1) the geometric complexity of the spinal column, (2) the variable locations of nerves, (3) the variable and intricate paths of nerves, and, (4) differences from person to person. Consequently, the target nerves are effectively invisible during the procedure.

For maximum effectiveness in single needle applications, it is generally desirable to position the single needle so that it lies parallel to the longitudinal axis of the targeted nerve fiber. Unfortunately, the structural complexity of the spine and the variable location of target nerves can sometimes limit the ability of a physician to manipulate and align a single needle in such a manner. Consequently, a physician must frequently make multiple placements of the single needle to ensure that the targeted fiber is adequately covered by lesions to sufficiently ablate the target nerves and treat the patient's pain.

The use of single needles is reasonably effective for certain applications such as spinal cord lesions. However, in most other spinal applications, a single needle application is more difficult to use and will not ensure adequate treatment at the target site. In particular, without multiple needle placements, single needle electrode methodology is substantially less effective for treating sympathetic ganglia, dorsal root ganglia, medial branch nerves to facets, and peripheral nerves, among others. For patient comfort and to expedite treatment, it is very desirable to have a system for radiofrequency treatment of spinal nerve tissue that minimizes the number of needle placements required to create an effective lesion.

General Methodology for RF Treatment

The objective of RF treatment in pain management is to effect pain fibers, which are nerve fibers known to conduct pain signals, in such a way as to interrupt transmission of pain signals from peripheral anatomy to the central nervous system. Such interruption will cause the pain experienced by the subject to lessen significantly. Radiofrequency treatment includes two primary approaches. The first, RF ablation, is a destructive methodology. The second, pulsed radiofrequency ablation, is considered a non-destructive treatment. Lesion, or destruction, of nerve tissue occurs above 44 degrees centigrade. Lesion produces a total disruption of sensory conduction. Pulsed RF produces a partial interruption of the sensory conduction. The RF effects are both time and temperature dependent.

Terms such as lesion, ablation, neurolysis and neurotomy are often used synonymously. Some chronic pain syndromes that may be treated by RF include CRPS, cervicogenic headaches, trigeminal neuralgia, occipital headaches, cancer pain, neck pain, low back pain, chest wall pain, post herniorraphy pain, sacroiliac joint pain, foot pain, and facet mediated pain. Facet mediated pain is well documented to occur especially after whiplash injury. In addition, facet mediated pain is also frequently associated with arthritis to the facet joints, which may not necessarily be secondary to whiplash.

For successful application of radiofrequency technology to treatment of spinal pain, several steps are required. First, one must determine the peripheral source of pain, such as a facet joint. Injections of local anesthetics assist in the diagnosis and identification of the peripheral pain source. If anesthetic is injected at the location of a suspect pain fiber, the pain sensed by the patient should subside to some degree noticeable to the patient.

Once the target fiber generating the peripheral source of pain has been diagnostically identified, a physician will prepare a plan and procedure for attempting to safely direct the percutaneous RF delivery device, a needle, to be contiguous to the target nerve. In single needle delivery systems, alignment in close proximity to the target nerve is essential to successful outcomes since the size of the generated lesion is generally limited by the size of the needle.

Access by the physician to the target site is dictated by anatomical position of the target nerve fiber. Traditionally, in the treatment of spinal pain, a curved needle, usually 22, 20 or 18 gauge, facilitates directional placement of the needles. X-ray fluoroscopy is generally used to assist in needle placement. However, multiple two-dimensional fluoroscopic views are necessary to maximize the probability for accurate needle placement.

Consequently, a repetitive pattern of directing and repositioning the needle using fluoroscopic X-ray imaging is required. In addition, frequent repositioning of the patient for adequate visualization using fluoroscopy may be required. These repetitive patterns tend to increase patient discomfort, increase the potential for harmful needle placement and extend the time and consequently the exposure to harmful x-rays required to provide the treatment. In particular, the treating physician, dealing with a number of patients, will normally suffer a much higher x-ray exposure from the fluoroscopy than any individual patient, thus increasing the likelihood of potentially negative future consequences for the physician.

Currently available pain management treatment systems and methods are inadequate. Procedures can be quite uncomfortable, sporadically effective and very costly. A patient can be subjected to several hours of applied repetitive procedures. The high cost and limited effectiveness limits the availability of the treatment to a large portion of the population. Additionally, it generally impedes a physician's ability to obtain approval for payment for the procedures from insurance providers.

Further, direct, real-time visualization of needle position and navigation, without dangerous x-ray exposure from fluoroscopy, is a highly desirable, but currently unavailable, technological capability. Also, three-dimensional visualization is very desirable, but generally unavailable. These capabilities would optimize the treatment procedure and improve the quality of care.

Once nerves are located, or presumed to be approximated, and a needle is placed contiguous to the nerves, a radio frequency generator delivers a measured band of radio waves to cause the target tissue to reach a temperature between 75 to 90 degrees Centigrade for duration of 60 to 90 seconds to create a discrete destructive lesion. Alternatively, the target tissue is raised to a temperature between 42 to 44 degrees Centigrade for a minimum of 120 seconds for a non-destructive treatment.

Various patents describe devices and methods for ablating tissue at various anatomical sites using radiofrequency energy. Others describe in greater detail variations in the design and operation of needles associated with delivering one or more electrodes of some type to a target ablation site.

Based on limitations described in the prior art, a radiofrequency ablation apparatus having simultaneous rotational, linear and flaring transverse deployment of one or more tines or electrodes in conjunction with optical imaging means and ultra-wide band tine locating and imaging means would be highly desirable. It would be further desirable for such a device able to facilitate ablation of spinal pain according to the various methods and at the plurality of locations described herein.

### SUMMARY OF THE INVENTION

A spinal nerve tissue ablation apparatus includes a stylet needle with a distal end having a rounded blunt tip sufficiently sharp to penetrate tissue but sufficiently blunt to avoid impinging on bony surfaces of spinal vertebra. The apparatus includes an energy delivery device having at least a first electrode and a second electrode. Each electrode has a tissue piercing distal end and is positionable in the stylet as the stylet is advanced through tissue. The first and second electrodes are deployable with curvature from the stylet. The stylet includes rotational means for orienting the first and second electrodes and directing the extension according to the curvature of the electrodes. The stylet further includes an optical imaging module to provide continual progressive feedback of ablation surface development. The apparatus further includes an ultra-wide band radio frequency scanning device capable of accurately determining the location of the electrodes within the spinal structure.

According to the present invention, an ablation apparatus, hereinafter the VIPER, has at least two conductive tines. Each tine has a tissue piercing distal end and is positionable in a compacted configuration within an elongate cylindrical stylet. Each tine is manufactured of material, such as surgical stainless steel, which causes the tine to return to a specific shape when deployed from the stylet. The tines are shaped and configured to resemble the fingers of a grasping, out-stretched hand when deployed out the end of the stylet.

The VIPER's ability to linearly vary the length of the tines, to fan and flare the tines, and to rotate the tines causes a much larger ablation area at a tissue site to be affected, when compared to prior art single needle solutions. For example, the footprint of the active area of a single needle in a single placement is 10 square millimeters (rectangular footprint with 10 millimeter maximum length by 1 millimeter wide); the available footprint of the active area of the VIPER in a single placement is approximately 315 square millimeters (circular with 10 millimeter diameter=3.14×10×10). The VIPER has the capability to affect thirty times the active area, when compared to a single needle. Use of the VIPER thereby dramatically increases the effectiveness of any tissue ablation treatment procedure.

The stylet of the VIPER is shaped to maximize the navigability of the VIPER through tissue and about bone and other surfaces. The stylet of the VIPER includes a distal curved forward jaw portion through which the tines of the VIPER extend when deployed. The jaw includes a blunt chin having a sharp leading edge or lip. With the tines in a compacted configuration, completely withdrawn into the VIPER stylet, a physician advances the stylet and tines through tissue until the jaw of the VIPER is maneuvered sufficiently close to the target tissue site to bring the tines in close proximity to the target tissue ablation site.

Once the jaw of the VIPER has been properly positioned, the physician then deploys the tines from the throat of the stylet toward the target tissue site. The tines are deployed in parallel flaring curvature, in the manner of a curved fan, thereby describing a somewhat linear path at the point where the distal ends of the tines contact the target tissue site.

In a preferred embodiment, all tines are deployed, rotated and retracted in unison. As the tines are deployed out the throat of the stylet of the VIPER, they flare outward from their compacted configuration to create a substantially larger lesion area, compared to the very narrow width of standard ablation needles. The further the tines are deployed out the throat of the stylet, the more the tines flare away from each other, creating a greater departure distance between the ends of the tines. In this manner, the VIPER is able to create lesions substantially longer than those normally created using only single needle ablation techniques.

In addition to providing linearly variable, flaring tine deployment, a physician is able to rotate the tines 360 degrees about a central axis of the stylet, thereby, allowing an even larger tissue area to be ablated without having to reposition the stylet of the VIPER.

In other embodiments, the VIPER provides for selectable and individual deployment of each tine which, depending on the number of tines provided in the VIPER stylet, allows for greater control of the actual shape of the lesion area.

One skilled in the art will recognize that the VIPER provides a plurality of additional deployment configurations, depending on the number of tines provided in the VIPER and the desire by a user to operate any number of tines either in unison or individually. Irrespective of the number of tines and their selectability, the VIPER provides for simultaneous rotation of all tines, independent of whether the tines are individually linearly deployed or simultaneously deployed in combination with other tines.

According to a first preferred embodiment of the present invention, two tines are deployed at a target site to delivery energy in a bipolar manner to ablate the target nerve tissue. A first energy delivery tine delivers energy to the target tissue. The delivered energy travels along and about the target tissue toward a second opposing receptor or grounding tine. As the energy travels through the tissue, the tissue is heated and changes state. Energy continues to be delivered to the tissue until the tissue has reached the desired temperature. The path described by the energy as it travels from the electrode tine to the receptor tine will result in a lesion of the target tissue. The length of deployment of the tines will determine the concentration of energy across a certain distance, and hence, will also determine the ultimate length of the lesion itself. The ability to vary the deployed length of the tines allows the physician to vary the size of the lesion as required to maximize effectiveness for a particular treatment protocol.

Additionally, using the rotational capability, a physician may rotate the tines of the VIPER to reach out to different tissue areas without having to remove the stylet from the patient. Consequently, the physician has substantially more control in designing procedures to create specific shapes and sizes of lesions.

Further, for various levels of energy applied to a target site, the ultimate shape of a created lesion will depend on the number and function of tines deployed, the deployment length of each tine, and, the selection of specific tines as either energy delivery electrodes or energy receptor electrodes. A first embodiment of the VIPER contemplates bipolar operation where at least one tine functions as a grounding or receptor tine and the other is used as an energy delivery tine. However, the VIPER may also be used in a monopolar mode where a dispersion grounding pad is applied to an external location on the patient and one or more of the VIPER tines are used for energy delivery to the target tissue. Monopolar operation of the VIPER generally causes lesions to develop in a manner different from that experienced with bipolar operation.

In a further embodiment, the VIPER includes three tines to create lesions of greater length than that created using only two tines. Where three tines are used, the two outside tines are shaped to flare further than just two tines. The two outside tines are energy delivery tines and the one central tine is a receptor or grounding tine. Alternatively, the one central tine may function as the sole energy delivery tine with both outside tines functioning as receptors.

Where three tines are deployed, the distance between an electrode tine and a receptor tine is no more than the distance between a single electrode and receptor tine used in the VIPER having just two tines. Consequently, the energy delivered by each outer electrode tine is more likely to be controlled and directed along a path toward the receptor tine. If distances between tines become too great, there may be sufficient resistance in the tissue to prevent the energy from traveling along the desired course to its associated receptor tine.

As with two tines, all three tines are advanceable with variable grasping curvature from the stylet of the VIPER to a tissue site. A physician is able to rotate the three tines about a central axis to provide greatest flexibility to the treating physician in designing a lesion program that will maximize effectiveness.

One skilled in the art will also recognize that, in addition to using tines for delivery or reception of energy to and through a target tissue site, the VIPER permits tines to be used for other purposes, particularly sensing. Various types of sensors may be incorporated with a tine to provide for optical, thermal or other sensing capability. Sensing provides feedback during the ablation process for monitoring and controlling the creation of the desired ablated lesion.

### OBJECTS OF THE INVENTION

Accordingly, the present invention is intended to satisfy several objectives.

A first object of the invention is to provide a spinal nerve tissue ablation apparatus that allows easier navigation of the spinal structure to create more effective ablative lesions at selected anatomical sites.

A second object of the invention is to provide a spinal nerve tissue ablation apparatus having optical imaging capabilities which allow incremental visual monitoring of tissue to allow for monitoring the accurate placement of the apparatus relevant to the target tissue site, to assist with verifying the effectiveness of the lesion treatment and to help avoid lesioning of inappropriate tissue which could harm the patient.

A third object of the invention is to provide a tissue ablation apparatus whose location can be accurately determined using ultra-wide band scanning techniques.

A fourth object of the invention is to provide a tissue ablation apparatus that allows in situ re-orientation of an energy delivery device adjacent target tissue without re-orienting an entire apparatus, thereby minimizing pain and discomfort to a subject patient and damage to inappropriate tissue.

A fifth object of the invention is to provide a tissue ablation apparatus that has at least two tines which are deployable from a stylet with curvature and a third tine which is separately deployable with equivalent or different curvature.

A sixth object of the invention is to provide a tissue ablation apparatus with selectively deployed tines.

A seventh object of the invention is to provide a tissue ablation apparatus that is configured to deploy tines selectively at a tissue site to create a desired tissue ablation lesion.

An eighth object of the invention is to provide a tissue ablation apparatus that can be inserted perpendicular, rather than parallel, to a nerve to be lesioned but still create a lesion of sufficient length to provide effective pain treatment to the patient.

A ninth object of the invention is to provide a tissue ablation apparatus with visual indicators that provide feedback to a treating physician regarding his or her manipulation of the tool.

These and other objects of the invention are achieved in a tissue ablation apparatus that includes a stylet with a distal end sufficiently sharp to penetrate tissue, stylet two or more tines, and providing for variable linear, rotational and flaring placement of the tines at a target tissue ablation site.

### BRIEF DESCRIPTION OF THE FIGURES

FIG. 1 is a view in perspective of the VIPER with the tines fully retracted, according to an embodiment of the invention.

FIG. 2 is a view in perspective of the VIPER having two tines, with both tines fully deployed, according to an embodiment of the invention.

FIG. 3 is a cross-sectional view of the VIPER spinal nerve tissue ablation apparatus of FIG. 2 taken along the lines 3-3.

FIG. 4 is an end view of a section of the stylet of the VIPER of FIG. 3 taken at the plane 5-5.

FIG. 5 is a side view of a section of the stylet of the VIPER of FIG. 3 taken at 5-5.

FIG. 6 is a perspective view of the forward portion of the VIPER having two deployed tines, according to an embodiment of the present invention.

FIG. 7 is a perspective view of the forward portion of the VIPER having three deployed tines, according to an embodiment of the present invention.

FIG. 8 is a perspective view of the forward portion of the VIPER having three deployed tines and a fourth central optical fiber, according to an embodiment of the present invention.

FIG. 9 is a close-up perspective view of the end of a VIPER having three tines, illustrating the linear and rotational variability of the VIPER, according to the present invention.

FIG. 10 is a top view of a blow-up of the tip of the VIPER having two deployed tines, according to the present invention.

FIG. 11 is a side view of the tip of the VIPER shown in FIG. 10.

FIG. 12 is a top view of a blow-up of the tip of the VIPER having three deployed tines, according to the present invention.

FIG. 13 is a side view of the tip of the VIPER shown in FIG. 12.

FIG. 14 is a side view of a conductive tine with marker, according to the present invention.

FIG. 15 is a side view of a sensor tine, according to the present invention.

FIG. 16 is a side view of an optical fiber tine, according to the present invention.

FIG. 17 is a side view of a dual-use tine having a conductive coating and core optical fiber, according to the present invention.

FIG. 18 is view in perspective of a typical use of both a two tine and three tine VIPER, according to the present invention.

### DETAILED DESCRIPTION

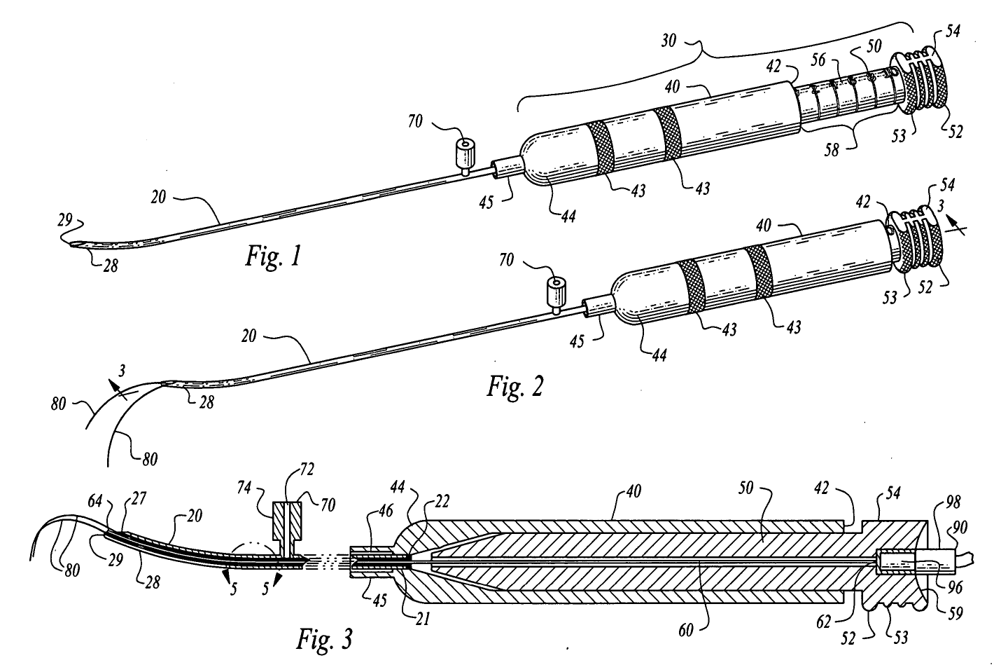

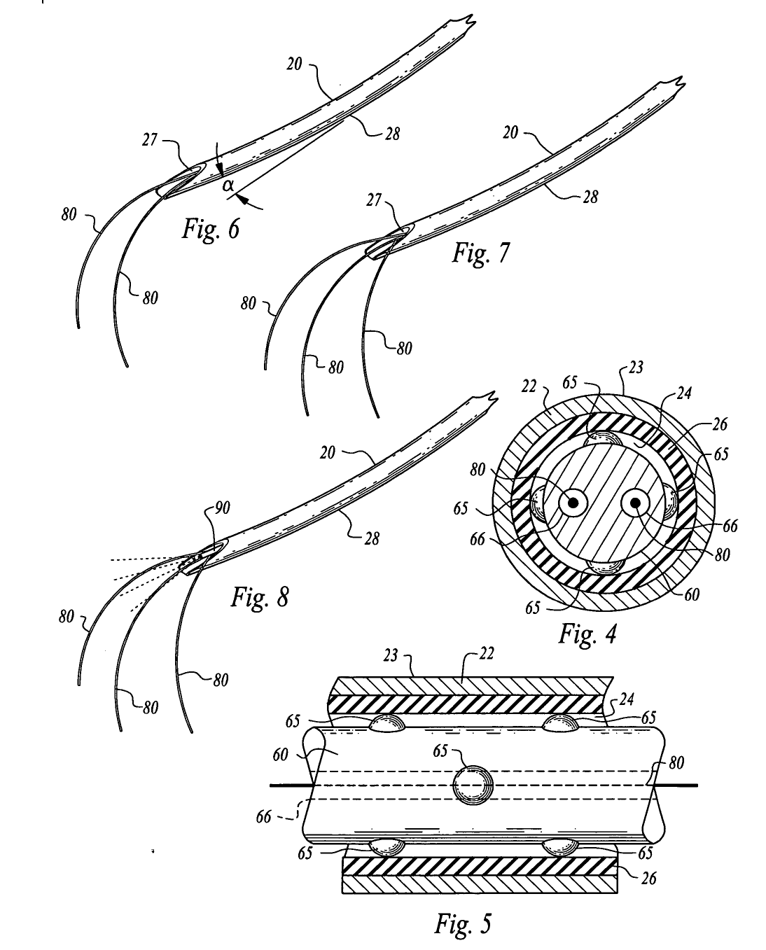

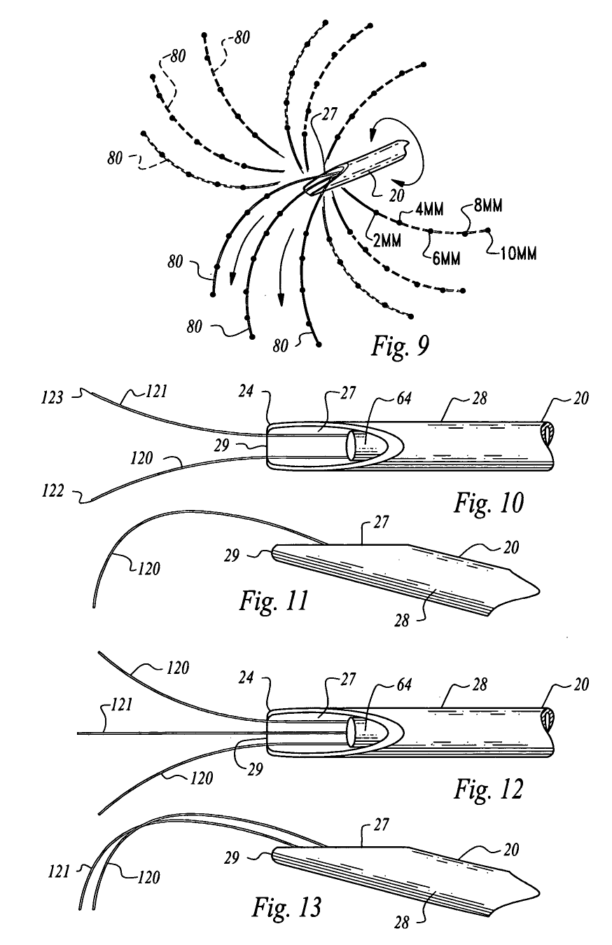

Referring to FIG. 1 and FIG. 2, a tissue ablation apparatus 10, hereinafter, the VIPER 10 includes a cylindrical tubular stylet 20. The stylet 20 is connected to a proximal control assembly 30. The control assembly 30 includes a female housing 40 and a male linear/rotational control member 50. The control member 50 is connected to a mandrel 60. The mandrel 60 is slidably and rotatably received within the stylet 20. A side infusion port 70 is disposed on a side of the stylet 20. One or more tines 80 are disposed and captured within the mandrel 40. The tines 80 are connected to the control member 50 of the control assembly 30. The control member 50 is further electrically and optically connected to an external system monitoring and control unit.

In further detail, the VIPER 10 includes a distal cylindrical elongate tubular stylet 20 fixably attached to a proximal control assembly 30. The stylet 20 is sufficiently small and made of sufficiently rigid material, such as surgical stainless steel, to allow the stylet 20 to penetrate skin and percutaneous tissue of a patient while minimizing trauma to surrounding tissue. The control assembly 30 is comprised of a female housing 40 and a linear/rotational control member 50. The control member 50 is attached to an elongate, cylindrical mandrel 60. The mandrel 60 is sized to slidably and rotatably mate within the stylet 20. The mandrel 60 is affixed to the control member 50 such that the mandrel 60 may be reciprocated or rotated within the stylet 20 by manipulation of the control member 60. Tines 80 are encapsulated within the entire length of the mandrel 60. The tines 80 are fixably attached to the connector 90 in the control member 50. The tines 80 have sufficient length to extend through the mandrel 60 and out the stylet 20. An infusion port 70 is disposed on the stylet 20 for delivery of various fluidic media to a target treatment site.

Referring now to FIG. 4 and FIG. 5, and still in greater detail, the VIPER 10 includes a stylet 20. The stylet 20 is a circular elongate tubular element having a cylindrical wall 22 with a smooth exterior surface 23 and an inner cylindrical bore 24. The bore 24 provides for passage of fluidic media through the annulus formed between the stylet 20 and the mandrel 60. The stylet 20 is made of surgical steel or other sufficiently rigid material such as carbon composite, plastic or acrylic so that a treating physician may insert the VIPER 10 to penetrate through skin, muscle, fat, connective tissue or other tissue, while minimizing patient discomfort.

Referring to FIG. 3, a proximal tail portion 21 of the stylet 20 is received and captured in the proximal end 44 of the control assembly housing 40. A seal 22 at the tail 21 of the stylet 20 encloses the mandrel 60 to minimize leakage of fluid back into the housing 40 while still allowing the mandrel 60 to slidably reciprocate. A ferrule 46 captures the stylet 20 in a nose piece 45 of the housing 40.

A distal curved forward portion 28 of the stylet 20 includes a rounded, but sharp nose 29. The nose 29 has sufficient sharpness to penetrate tissue. The curved forward portion 28 in conjunction with the nose 29 improves a user's ability to navigate the stylet 20 of the VIPER 10 along bone to a target tissue site without having the nose 29 inadvertently impinge on bony surfaces while being manipulated to reach the target site. The curved forward portion 28 includes a throat 27.

The curved forward portion 28 of the stylet 20 may have different shapes to control the shape of deployment of the tines 80, and, to adjust the navigation characteristics when manipulated by a physician. For example, the forward portion 28 may be circular, oval or flat to control and adjust the deployment configuration of the tines 80.

Referring once again to FIG. 1, the VIPER 10 includes a control assembly 30. The control assembly 30 is comprised of a female housing 40 and an integrated rotational and linear control member 50. The female housing 40 is sized to snugly, but slidably and rotatably receive the control member 50. The housing 40 includes knurled surfaces 43 on its exterior for improving the grip of the physician while manipulating the VIPER 10 to reach a target tissue site in a patient. The housing 40 has a proximal end 42 and a distal end 44. The distal end 44 is closest to the stylet 20.

Referring to FIG. 3, the control member 50 is fixably attached to the mandrel 60. The control member 50 is rotated by the physician to rotate the mandrel 60 about its central axis. The control member 50 includes a proximal knob end 52 which is grasped and manipulated by the physician to adjust the rotational placement of the mandrel 60, and concurrently, the orientation of the tines 80. The control knob end 52 further includes an indicator surface portion 54 which coincides with the directional curvature of the tines 80. As a physician rotates the mandrel 60 and tines 80 by manipulating the control knob 52, the indicator surface 54 will provide a tactile and visual indicator of the deployment direction of the tines 80. Although shown here as a flat surface, the indicator surface 54 on the control knob 52 may also be shaped as a protrusion, indentation, visual line or other similar tactile and visual indicator. The control knob 52 also includes a knurled exterior surface 53 to enhance the physician's grip of the knob 52 during operation.

Referring to FIG. 1, a neck portion 56 of the control member 50 includes a graduated scale 58. The scale 58 provides multiple markings along its length providing a visual indicator of the length of deployment of the tines 80 out the throat 27 of the stylet 20. When the control member 50 is fully linearly retracted, as shown in FIG. 1, the number marking “0” on the scale 58, will show just adjacent the proximal end 42 of the housing 40. The number marking “0” indicates that the tines 80 are not deployed out the throat 27 of the stylet 20. When the control member 50 is fully inserted into the housing 40, as shown in FIG. 2, the number marking “10” on the scale 58 will show just adjacent the proximal end 42 of the housing 40. The “10” indicates that 10 millimeters length of the tines 80 are fully deployed out the throat 27 of the stylet 20. Although shown as providing variable tine length between 0 and 10 millimeters, the graduated scale 58 may be changed to coincide to any available deployable length of the tines 80.

Near the distal end of the control assembly housing 40, an infusion port 70 protrudes from the stylet 20. The infusion port 70 provides a means for delivering fluidic media down the stylet 20 to a target tissue site. Disposed adjacent the distal end 44 of the control housing 40, the infusion port 70 includes a channel 72 which provides a conduit for delivering fluids to the bore 24 of the stylet 20. The infusion port 70 includes connecting means 74 which allow a syringe to be attached to the infusion port 70 such that fluidic media can be easily delivered through the port 70. Although not shown in the present drawings, the infusion port 70 may also include a valve assembly, such as a check valve, disposed within the channel 72 to control fluid flow or prevent backflow of bodily fluids through the stylet 20 and out the infusion port 70.

Referring to FIG. 3, the control assembly 30 further includes a conductive and optical connector 90 centrally disposed in a recess 59 of the control member 50.

Referring now to FIG. 4 and FIG. 5, a slidable and rotatable mandrel 60 is snugly but movably disposed within the bore 24 of the stylet 20. The mandrel 60 is preferably made of Teflon or other similar material to provide maximum lubricity and minimal friction when reciprocated within the stylet 20. With reference to FIG. 3, the mandrel 60 includes a proximal tail 62 and a distal tongue 64. The tail 62 is fixably attached to the control member 50. The tongue 64 is disposed toward the throat 27 of the stylet 20 and lies adjacent the throat 27 of the stylet 20 when the tines 80 are fully deployed. Referring again to FIG. 4 and FIG. 5, one or more individual lumens 66 penetrate and run the length of the mandrel 40. The lumens 66 run parallel to a central axis of the mandrel 40. The lumens 66 are sized to both fixably and slidably receive individual tines 80. The mandrel 60 is rotatable within the stylet 20 so as to cause tines 80 captured within the mandrel 60 to also be rotated in unison about the central axis of the mandrel 60. The mandrel 60 may be rotated with tines 80 in either: 1) a fully withdrawn compacted configuration, 2) a fully deployed state, or 3) any position between fully withdrawn or fully deployed. In most cases, it will be preferable to rotate the mandrel 60 only when the control member 50 is fully retracted, as shown in FIG. 1, with the tines 80 fully withdrawn into a compacted configuration within the stylet 20.

Referring to FIG. 4, a plurality of centralizing, spacer protrusions 65 extend outwardly from the mandrel 60. The spacer protrusions 65 maintain the orientation of the mandrel 60 in a centralized position in the bore 24 of the stylet 24. The protrusions 65 also maintain an open fluid pathway through the bore 24 of the stylet 20 through which fluidic media may be delivered to a target tissue site.

In a separate embodiment, not shown here, the mandrel 60 is enclosed by an external skin to increase rigidity and hardness. The external skin either partially or completely encloses the mandrel. The skin may be made of an external steel tube, Kevlar fiber, or other similar material.

Referring now to FIG. 6, FIG. 7 and FIG. 8, three different tine configurations are illustrated. FIG. 6 is an illustration of a VIPER 10 having two tines 80; FIG. 7 is an illustration of a VIPER 10 having three tines 80; FIG. 8 is an illustration of a VIPER 10 having three tines 80 and a separate optical fiber 90.

In FIG. 6, the VIPER 10 with two deployed tines 80 is illustrated. The VIPER includes a stylet 20 having a forward curved portion 28. The forward portion 28 of the stylet 20 will deviate from the central longitudinal axis of the VIPER by an angle, α. The angle, α, can be varied between one and thirty degrees, depending on the curvature most suited to the particular application. In most spinal applications, it is anticipated that an angle α between five and twenty degrees would be most suitable.

Referring to FIG. 7, the VIPER 10 with three deployed tines 80 is illustrated. Three tines 80 will provide for greater lesion coverage than two tines 80 since the distance between the two outside tines 80 is greater where three tines 80 are deployed.

Referring to FIG. 8, the VIPER 10 having three deployed tines 80 and a separate optical fiber 90 is illustrated. The optical fiber 90 provides a means for both shining light of various frequencies on the target tissue, and, for collecting light reflected back by the target tissue. The reflection of light from the target tissue will provide a mechanism for both identifying the type of tissue adjacent the nose 29 of the VIPER 10, and, for tracking the progress of tissue ablation. Ablation progress may be measured by changes in reflectance, absorptance, and fluorescence of the target tissue. Optical signals collected by the optical fiber 90 are processed by a monitoring and control unit to assess the level of ablation.

Referring again to FIG. 3, the control member 50 is fixably attached to the proximal tail 62 of the mandrel 60. The control member 50 provides for both linear and rotational manipulation of the mandrel 60 and tines 80.

Now, referring to FIG. 9, the control member 50 may be manipulated by the physician to rotate the entire mandrel 60 three hundred sixty degrees about a central axis of the mandrel 60. The rotation of the mandrel 60 simultaneously provides for re-orientation of the tines 80 out the throat 27 of the stylet.

In addition, the control member 50 is used to adjust the length of deployment of the tines 80. Referring once again to FIG. 9, the length of the tines 80 may be adjusted by the physician by inserting or withdrawing the control member 50 in the housing 40. For example, the tines 80 are shown in six different states of deployment: 0 mm, 2 mm, 4 mm, 6 mm, 8 mm and 10 mm. These deployment positions correspond to the gradation markings on the scale 58 on the neck 56 of the control member 50.

Referring once again to FIG. 1 and FIG. 2, a knurled rotational control surface 53 is provided on the control knob 52 of the control member 50. The control surface 53 is twisted by the treating physician to change the orientation of the mandrel 60 and tines 80 within the stylet 20. The control knob 52 also includes a smooth indicator surface 54. The knurled surface 53 provides a better grip for the physician while manipulating the control member 50. The smooth surface indicator 54 provides tactile and visual indication of tine 80 orientation.

In a separate embodiment not shown here, the control assembly 30 includes a separate slidable inner linear control element. The inner linear control element is centrally disposed within a square recess of the control member 50. The inner linear control element is shaped to immovably mate with the square recess such that the slidable linear control element rotates in unison with the mandrel 60 whenever the control member 50 is adjusted by the physician. The tines 80 are fixably connected to the inner linear control element. Thus, whenever the physician elects to reorient the tines 80 about the central axis, the control member 50, the inner linear control member, the mandrel 60 and the tines 80 all rotate in unison. Optical and conductive connectors 90 are centrally disposed in the distal end of the inner linear control element. A distal end of the linear control element is mechanically, optically and conductively connected to one or more tines 80.

Again, although not shown here, in a further embodiment, both the control member 50 and the inner linear control element include a movement translation means which causes the linear motion applied to the tines 80 by movement of either the control member 50 or the inner linear control element to be reduced, providing greater sensitivity and control for the treating physician. Hence, for example, in one configuration, a 1 centimeter linear movement of the control member 50 by the treating physician would produce only a 1 mm linear movement of the tines 80. One skilled in the art will recognize that the movement translation means of the VIPER 10 allows the ratio of linear movement of either the control member 50 or the inner linear control element to the linear movement of the tines 80 to be adjusted.

Referring to FIG. 10 and FIG. 11, the forward portion 28 of the VIPER 10 is illustrated, with two conductive tines 120, 121 deployed out the throat 27 of the VIPER 10. The stylet 20 includes the forward curved portion 28 having a throat 27. The stylet 20 further includes a distal end 24. A blunt, but sharp, nose 29 is located on the distal end 24 of the stylet 20. In a fully deployed state, the tongue 64 of the mandrel 60 is located adjacent the throat 27 of the stylet 20. A conductive electrode energy delivery tine 120 having a sharp distal end 122 delivers energy to the target tissue area. A receptor or grounding electrode tine 121 having a sharp distal end 123 receives energy through the target tissue from the delivery tine 120. The delivery tine 120 and receptor tine 121 flare outwardly away from each other when deployed out the throat 27 of the VIPER 10. The tines 120, 121 are positioned in the mandrel 60 so that they are electrically isolated. The tines 120, 121 deploy outwardly and downward from the throat 27 of the VIPER 10.

Referring to FIG. 12 and FIG. 13, the forward portion 28 of the VIPER 10 is illustrated, with three conductive tines 120, 121 deployed out the throat 27 of the VIPER 10. The stylet 20 includes the forward curved portion 28 having a throat 27. The stylet 20 further includes a distal end 24. A blunt, but sharp, nose 29 is located on the distal end 24 of the stylet 20. In a fully deployed state, the tongue 64 of the mandrel 60 is located adjacent the throat 27 of the stylet 20. A first and second conductive electrode energy delivery tine 120 having a sharp distal end 122 delivers energy to the target tissue area. A receptor or grounding electrode tine 121 having a sharp distal end 123 receives energy through the target tissue from the first and second delivery tines 120. The energy delivery tines 120 flare outwardly away from each other and the centrally located receptor tine 121 when deployed out the throat 27 of the VIPER 10. The tines 120, 121 are positioned in the mandrel 60 so that they are electrically isolated. The tines 120, 121 deploy outwardly and downward from the throat 27 of the VIPER 10.

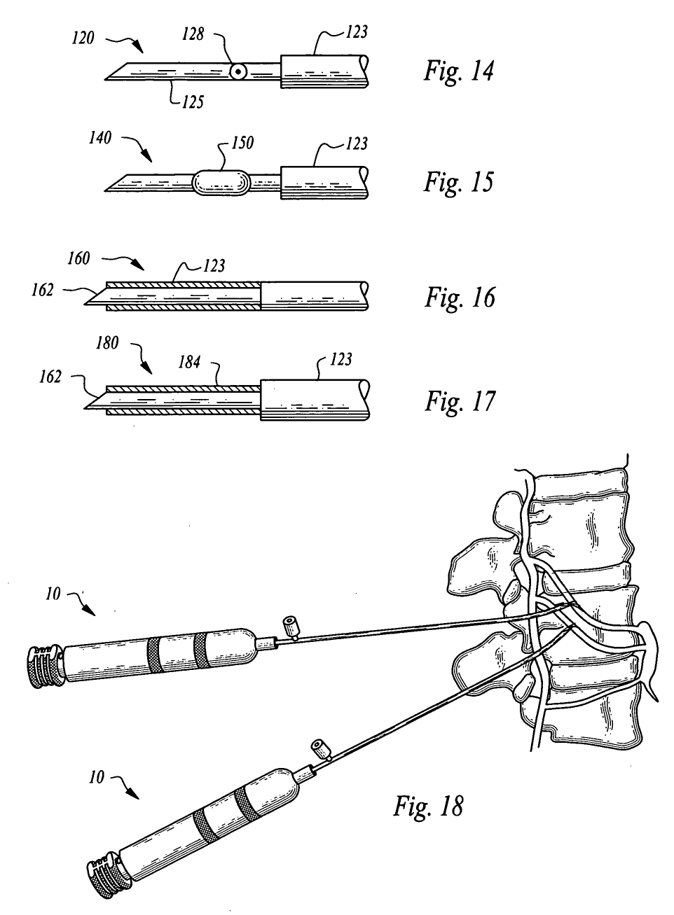

Referring now to FIG. 14, FIG. 15, FIG. 16 and FIG. 17, various types of tines 80 are illustrated, all of which may be used in different embodiments of the VIPER 10.

FIG. 14 illustrates the composition of both an energy delivery tine 120 and a receptor tine 121. The energy delivery tine 120 is equivalent in construction to an energy receptor tine 121. The tine 120, 121 include an insulating portion 123 and a conductive/receptive portion 125. The tines 120, 121 further include a marker 128. A core 122 of both the energy tine 120 and receptor tine 121 is made of a conductive material, such as copper, stainless steel, silver or other conductive material. Each tine 120, 121 is then coated along an insulated portion 123 of its length with Teflon or other similar insulating material. A distal radiating and conductive portion 125 of the tines 120, 121 is not coated with insulating material, thereby allowing energy to be delivered to a target site, or, energy to be received by an affiliated receptor tine 121 from a nearby energy tine 120, creating an ablated region in the target tissue.

FIG. 15 illustrates a sensor tine 140 having an embedded sensor 150. Sensor tine 140 is similar in construction to both the energy delivery tine 120 and the receptor tine 121 except that various types of sensors 150 are attached to the core 122 of the sensor tine 140.

FIG. 16 illustrates an optical tine 160 fully encased in an insulating and sealing layer 123. The optical tine 160 has an inner core 162 composed of optical fiber. The optical tine 160 is coated with an outer insulating layer 123 of Teflon or other similar material to both protect the optical fiber 162 and provide sufficient strength to the optical tine 160 to allow the optical tine 160 to sufficiently penetrate various tissue types.

FIG. 17 illustrates a hybrid electro-optical tine 180. The electro-optical tine 180 includes an optical fiber core 162. The optical core 162 is coated with a conductive layer 184. The electro-optical tine further includes an insulated portion 123. The hybrid optical/energy delivery/receptor tine 180 provides both energy delivery/reception capabilities and optical imaging capabilities. The conductive layer 184 is comprised of conductive material selected from the group including silver, gold, carbon, aluminum or other similar material capable of conducting various forms of energy including microwave, radiofrequency and electrical energy. The conductive layer 184 is partially coated with an insulating layer 123. The insulating layer 123 may be TEFLON or other similar coating material able to provide both insulating and lubricating capability.

Whenever it is desirable to produce a VIPER 10 with the ability to move individual tines 80, due to the small size of the lumens 66 of the mandrel 60 and the tines 80, all tine types including energy delivery 120, receptor 121, sensor 140, optical 160, and hybrid 180, can be coated with a low friction, lubricating insulating layer such as Teflon.

The optical fiber core 162 of the optical tine 160 and hybrid tine 180 provides light transmission and reception capability where light signals of varying frequencies may be applied to the target tissue and resulting signals relayed to a monitoring and control module. Light is transmitted through the optical fiber 162 and directed toward target tissue. As the target tissue is ablated, the optical transmissivity, absorptance and reflectance of the target tissue will change, changing the percentage of light that is absorbed or reflected. This change in the optical properties of the target tissue during ablation is caused by protein coagulation among other natural phenomena. The changes in optical transmissivity of nerve fiber are similar to the changes observed when frying an egg. At first, before heating, the white of the egg is transparent. As the egg white is heated, the normally transparent egg whites change color, from clear to white, due to coagulation of the protein in the egg whites. Light that would normally be absorbed by or transmitted through the egg white will be reflected as the egg white is heated and cooked, causing it to change color to white. Likewise, for nerve fiber, as the target tissue begins to heat and cook, the light applied to the tissues will begin to be reflected and picked up by the optical fiber 162 as ablation proceeds. A monitoring and control module correlates the reflected and scattered light to track the level of ablation of the target tissue.

Still referring to FIG. 17, the conductive layer 184 surrounding the optical fiber core 162 of the hybrid tine 180 delivers energy through the hybrid tine 180 to a target tissue site to ablate the target tissue.

Use and Operation of the VIPER

The following discussion describes various configurations and methods of use of the VIPER 10, using different tine 80 configurations.

Example applications and procedures which are believed to immediately benefit from use of the features of the VIPER are provided in the table below. Relevant Current Procedural Terminology (CPT) codes are also provided in the table for reference. CPT Codes describe medical or psychiatric procedures performed by physicians and other health providers. The codes are derived from the Health Care Financing Administration to assist in the assignment of reimbursement amounts to providers by Medicare carriers.

| AREA | TARGET | CPT CODE |
|---|---|---|
| FACET | Cervical | 64626/27 |
| | Thoracic | 64626/27 |
| | Lumbar | 64622/23 |
| SYMPATHETIC GANGLIA | Stellate | 64680 |
| | Celiac Plexus | 64680 |
| | Lumbar Paravertebral | 64680 |
| DORSAL ROOT GANGLIA | Cervical | 64640 |
| | Thoracic | 64640 |
| | Lumbar | 64640 |
| SACRAL ILIAC JOINT | | 64640 |
| INTRADISCAL | | 62292 |
| PERIPHERAL NERVES | Occipital Nerve Block | 64640 |
| | Intercostan Nerve Block | 64620/21 |
| | Ilioinguinal Nerve Block | 64640 |
| | Pudendal Nerve Block | 64630 |

First, a general description of the operation of the VIPER 10, where the VIPER 10 has already been manipulated to reach a target tissue site, is provided. For purposes of general description, operation of the VIPER 10 where two tines 80 are provided in the stylet 20 is described. However, the method of operation is equally applicable where more than two tines 80 are provided.

Referring to FIG. 18, an illustration of the use of both a two-tine 80 and three-tine 80 VIPER 10 to ablate nerve fibers running along the spinal column is provided.

Now, referring to FIGS. 9, 10, 11, 12 and 13, the following general description of use of the VIPER 10 describes such use where a first tine 80 is an energy delivery tine 120 and a second tine 80 is an energy receptor tine 121. The described operation is applicable to ablation of tissue at any site.

Once the VIPER 10 has reached the desired target tissue site, with the tines 120, 121 withdrawn in a fully compacted state, the treating physician can linearly and rotationally manipulate the VIPER mandrel 60 and tines 120, 121 using the control assembly 30 to provide a desired lesioning affect. In one method, the control member 50 is slowly slid toward the distal end 24 of the stylet 20 causing the compacted tines 120, 121 to move forward through the bore 24 of the stylet 20 and out the throat 27 of the stylet 20 toward a deployed state.

Also, referring to FIG. 1, to control the length of extension beyond the nose 29 of the stylet 20, a visual graduated indicator 58 on the control element 50 illustrates the length of the tines 120, 121 extended beyond the nose 29 of the stylet 20. Typically, the tines 120, 121 will be deployed in increments of 1 mm across distances of 1 to 20 mm.

In a first general method of use, the VIPER apparatus 10 includes two tines 80, a first energy delivery tine 120 and a second receptor tine 121. Energy tine 120 and receptor tine 121 are positionable in a compacted configuration within stylet 20 as stylet 20 advances through tissue. Energy tine 120 and receptor tine 121 have tissue piercing distal ends 122 and 123, respectively. Referring to FIG. 9 and FIG. 10, energy tine 120 and receptor tine 121 are deployed in combination with curvature from a distal end 24 of stylet 20 to a selected nerve fiber tissue site. In a further embodiment, not shown in the associated drawings, the tines 120, 121 may be individually and independently linearly deployed from the VIPER 10.

Energy tine 120 and receptor tine 121 are deployable to be controllably positioned at a desired location relative to the target tissue site that includes internal and external placement at a periphery of the target tissue site and at any desired location relative to the target tissue site. The selectable and variable deployment of energy tine 120 and receptor tine 121 can be achieved by varying the amount of advancement of energy tine 120 and receptor tine 121 from stylet 20, by independently advancing energy tine 120 and receptor tine 121 from stylet 20, by varying the lengths and/or sizes of energy delivery/reception surfaces 125 of energy tine 120 and receptor tine 121, by varying materials used for energy tine 120 and receptor tine 121, and, by varying the geometric configuration and shape of energy tine 120 and receptor tine 121 in their deployed states.

As illustrated in FIG. 1, tines 80 are in compacted positions within the stylet 20 with the control member 50 fully withdrawn. The VIPER 10 is kept in this position as the stylet 20 is navigated to a target tissue site. Referring to FIG. 2, as tines 80 are advanced from stylet 20, the tines 80 move to a deployed state from their compacted configurations.

As illustrated in FIG. 1 and FIG. 2, control member 50 includes a knurled, rotational control surface 53 which may be manipulated by the physician to rotate the mandrel 60 about its central axis. Rotation of the mandrel 60 and the tines 80 by the physician by rotational twisting of the proximal rotational control surface 53 of the control knob 50 allows re-orientation of the tines 80. The control knob 50 includes a directional indicator surface 54 which allows the physician to determine the orientation of the tines 80 and the direction of curvature of the tines 80 when deployed from the stylet 20 to an extended state. The control member 50 includes a graduated scale 58 along its neck 56 to provide a visual indicator to the physician indicating the length of extension of the tines 80.

When stylet 20 reaches a target tissue site, tines 80 are deployed from the throat 27 of stylet 20. In the deployed state, tines 80 flare from their compacted configuration in stylet 20 and are selectively positioned relative to tissue site by applying either rotation or linear translation of the tines 80. Tines 80 can be portioned within an interior of a tissue site, at the exterior of tissue site, as well as combinations thereof. Tines 80 are advanceable different lengths from distal forward portion 28 of stylet 20.

Volumetric spinal nerve tissue ablation can proceed from the interior or exterior of a target tissue site as well as various combinations thereof with each deployed tine 80 in order to create a selectable and predictable spinal nerve tissue ablation.

Referring now to FIG. 14, energy tine 120 and receptor tine 121 can be made of a variety of conductive materials, both metallic and non-metallic. One suitable material is type 304 stainless steel of hypodermic quality. In some applications, all or a portion of energy tine 120 and receptor tine 121 can be made of a shaped memory metal, such as nickel titanium. Further, a radiopaque marker 128 can be coated on energy tine 120 and receptor tine 121 for visualization purposes while the tines are embedded within a patient's tissue.

With reference to FIG. 9, the lengths of energy tine 120 and receptor tine 121 are varied to advance different distances from the distal forward portion 28 of stylet 20. The lengths can be determined by the actual physical length of energy tine 120 and receptor tine 121, the length of an energy delivery surface of energy tine 120 and receptor tine 121 and the length of energy tine 120 and receptor tine 121 that is not covered by an insulator. Suitable lengths include, but are not limited to 5 mm, 10 mm and 20 mm. The actual desired length of energy tine 120 and receptor tine 121 depends on the location and size of tissue site to be ablated.

With reference to FIG. 15, in a further configuration, an additional deployable sensor tine 140 can likewise be coupled to the control element 50. A sensor tine 140 provides a variety of different functions including but not limited to the placement of a sensor 150 at a selected tissue site to measure/monitor temperature and/or impedance. In this manner, temperature and/or impedance is measured or monitored at a distal portion of a target tissue site or at any position in or external to the targeted tissue site. The sensor tine 140 is deployable from the throat of stylet 20 with grasping, parallel curvature similar to that of energy tine 120 and receptor tine 121. The sensor tine 140 would preferably be deployed between the energy tine 120 and receptor tine 121, but may also be integrated with the energy tine 120 and receptor tine 121.

A sensor tine 140 permits accurate measurement of temperature at a target tissue site in order to determine, (i) the extent of nerve tissue ablation, (ii) the amount of nerve tissue ablation, (iii) whether or not further nerve tissue ablation is needed and (iv) the boundary or periphery of the ablated nerve tissue.

Sensors 150 incorporated with sensor tine 140 are of conventional design, including but not limited to thermistors, thermocouples, resistive wires, and the like. A suitable thermal sensor 150 includes a T-type thermocouple with copper constantene, J type, E type, K type, fiber optics, resistive wires, thermocouple IR detectors, and the like. It will be appreciated that sensors 150 need not be limited to a thermal sensor.

Sensor 150 measures temperature and impedance to permit monitoring and control of the desired level of spinal nerve tissue ablation to be achieved while avoiding the destruction of too much tissue. By monitoring the temperature at various points within and outside of the interior of the target tissue site, a determination of the selected tissue mass periphery can be made, as well a, a determination of when spinal nerve tissue ablation is complete. If at any time, a sensor 150 determines that a desired spinal nerve tissue ablation temperature is exceeded, then an appropriate feedback signal is received at a control and monitoring system coupled to the VIPER 10 which then regulates the amount of energy delivered to energy tine 120.

An energy source generator, integrated with a monitoring and control module, can be a radiofrequency power supply, an ultrasound energy source, a microwave generator, a resistive heating source, a laser and the like. Microwave antenna, optical fibers, resistive heating elements and ultrasound transducers can be substituted for energy tine 120 or receptor tine 121. When energy source is a radiofrequency power supply, 5 to 200 watts, 5 to 100, and still more preferably, 5 to 50 watts of electromagnetic energy is delivered from the energy source to the energy delivery tine 120 without impeding out the electrode 120.

Energy tine 120 and receptor tine 121 are typically electromagnetically coupled to the energy source. The coupling can be direct from energy source to each electrode tine 120, or indirect by using a collet, sleeve and the like which couples one or more electrodes to the energy source.

The mandrel 60 is disposed within the bore 24 of the stylet 20. The annulus formed by the mandrel 60 and stylet 20 provides a channel by which various fluidic media may be delivered to the target tissue area. The fluidic media are delivered to the annulus via the infusion port 70. Suitable fluidic media include but are not limited to electrolytic solutions, chemotherapeutic agents, drugs, medicaments, gene therapy agents, contrast agents and the like.

Tines 80 can have a variety of different geometric cross-sections. Tines 80 can be made of conductive solid or hollow straight wires of various shapes such as round, flat, triangular, rectangular, hexagonal, and, elliptical. In addition, tines 80 can be made of optical fiber coated with conductive material.

Each tine 80 has an exterior surface that is wholly or partially insulated and provides a non-insulated area which is an energy delivery surface 125.

An energy tine 120 and receptor tine 121 include insulation 123. The active area of energy tine 120 and receptor tine 121 is non-insulated and provides an energy delivery surface 125.

Referring to FIG. 9, tines 80 are selectably deployable from stylet 20 with similar curvature to create variable length and width of desired area of spinal nerve tissue ablation. The selectable deployment is achieved by having tines 80 with, (i) diverging curvatures, (i) different advancement lengths from stylet 20, (iii) selectable insulation provided at each of the deployed tines 80, or (iv) through axial rotation of the control assembly 30, causing variable rotational deployment of the tines 80.

Deployed tines 80 can create a variety of different geometric spinal nerve tissue ablation zones including but not limited to spherical, semi-spherical, spheroid, triangular, semi-triangular, square, semi-square, rectangular, semi-rectangular, conical, semi-conical, quadrilateral, semi-quadrilateral, semi-quadrilateral, rhomboidal, semi-rhomboidal, trapezoidal, semi-trapezoidal, combinations of the preceding, geometries with non-planar sections or sides, free-form and the like.

In another configuration of the VIPER 10, not illustrated in the accompanying drawings, during deployment of the tines 80 from the stylet 20 of the VIPER 10, the VIPER 10 includes a means for preventing rotation of the tines 80 of VIPER 10 just prior to extending beyond the distal tip 28 of the stylet 20 into the target tissue site. The rotational restriction means ensures that the deployed tines 80 do not cause unintended trauma to the target tissue site or any surrounding tissue.

A feedback monitoring and control system is connected to energy source, sensors 150 and tines 80. The feedback control system receives temperature or impedance data from sensors 150 and the amount of electromagnetic energy received by tines 80 is modified from an initial setting of spinal nerve tissue ablation energy output, spinal nerve tissue ablation time, temperature, and current density (the “Four Parameters”). The feedback control system can automatically change any of the Four Parameters. Feedback control system can detect impedance or temperature and change any of the Four Parameters. Feedback control system can include a multiplexer to multiplex different tines 80 and a temperature detection circuit that provides a control signal representative of temperature or impedance detected at one or more sensors 150. A microprocessor can be connected to the temperature control circuit.

The user of the VIPER 10 can input an impedance value which corresponds to a setting position located at VIPER 10. Based on this value, along with measured impedance values, feedback control system determines an optimal power and time need in the delivery of energy. Temperature is also sensed for monitoring and feedback purposes. Temperature can be maintained to a certain level by having feedback control system adjust the power output automatically to maintain that level.

In another embodiment, feedback control system determines an optimal power and time for a baseline setting. Ablation volumes or lesions are formed at the baseline first. Larger lesions can be obtained by extending the time of ablation after a center core is formed at the baseline. A completion of lesion creation can be checked by advancing tines 80 from the throat 27 of stylet 20 to a desired lesion size and by monitoring the temperature at the periphery of the lesion.

In another embodiment, feedback monitoring and control system is programmed so the delivery of energy to the energy delivery tines 120 is paused at certain intervals at which time temperature is measured. By comparing measured temperatures to desired temperatures feedback control system can terminate or continue the delivery of power to energy delivery tines for an appropriate length of time.

The following discussion pertains particularly to the use of an radiofrequency energy source and radiofrequency energy delivery tines but applies to other energy sources including but not limited to microwave, ultrasound, resistive heating, coherent and incoherent light, and the like.

Current delivered to energy delivery tines 120 is measured by a current sensor. Voltage is measured by voltage sensor. Impedance and power are then calculated at power and impedance calculation device. These values can then be displayed at a user interface and display. Signals representative of power and impedance values are received by the controller.

A control signal is generated by the controller that is proportional to the difference between an actual measured value, and a desired value. The control signal is used by power circuits to adjust the power output in an appropriate amount in order to maintain the desired power delivered at energy delivery tines 120.

In a similar manner, temperatures detected at sensors 150 provide feedback for determining the extent of spinal nerve tissue ablation, and when a completed spinal nerve tissue ablation has reached the physical location of sensors 150. The actual temperatures are measured at temperature measurement device and the temperatures are displayed at a user interface and display. A control signal is generated by the controller that is proportional to the difference between an actual measured temperature, and a desired temperature. The control signal is used by power circuits to adjust the power output in an appropriate amount in order to maintain the desired temperature delivered at the respective sensor 150. A multiplexer can be included to measure current, voltage and temperature, at the numerous sensors 150, and energy is delivered to energy delivery tines 120. A variable electrode tine setting is coupled to the controller.

A controller can be a digital or analog controller, or a computer with software. When a controller is a computer, it can include a CPU coupled through a system bus. On this system can be a keyboard, a disk drive, or other non-volatile memory systems, a display, and other peripherals, as are known in the art. Also coupled to the bus are a program memory and a data memory.

User interface and display includes operator controls and a display. A controller can be coupled to imaging systems, including but not limited to ultrasound, CT scanners, X-ray, MRI, mamniographic X-ray and the like. Further, direct visualization and tactile imaging can be utilized.

Ultra-wide Band Device Positioning—In another embodiment, a controller is coupled to an imaging system using ultra-wide band, hereinafter, UWB, radio frequencies for imaging the spinal area and the location of the tines 80. During operation, the tines 80 are impressed with a signal that causes the tines 80 to be readily identifiable by the UWB scanning and imaging device.

The output of current sensor and voltage sensor is used by the controller to maintain a selected power level at energy delivery tines 120. The amount of radiofrequency energy delivered controls the amount of power. A profile of power delivered can be incorporated in a controller, and a preset amount of energy to be delivered can also be profiled.

Circuitry, software and feedback to a controller result in process control, and the maintenance of the selected power, and are used to change, (i) the selected power, including radiofrequency, microwave, laser and the like, (ii) the duty cycle (on-off and wattage), (iii) bi-polar or mono-polar energy delivery and (iv) infusion medium delivery, including flow rate and pressure. These process variables are controlled and varied, while maintaining the desired delivery of power independent of changes in voltage or current, based on temperatures monitored at sensors 156.

Various sensors 150, including a current sensor and voltage sensor are connected to the input of an analog amplifier. The analog amplifier can be a conventional differential amplifier circuit for use with sensors 150. The output of the analog amplifier is sequentially connected by an analog multiplexer to the input of A/D converter. The output of analog amplifier is a voltage which represents the respective sensed temperatures. Digitized amplifier output voltages are supplied by an analog to digital converter to a microprocessor. Any suitable microprocessor or general purpose digital or analog computer can be used to calculate impedance or temperature.

The microprocessor sequentially receives and stores digital representations of impedance and temperature. Each digital value received by the microprocessor corresponds to different temperatures and impedances.

Calculated power and impedance values can be indicated on a user interface and display. Alternatively, or in addition to the numerical indication of power or impedance, calculated impedance and power values can be compared by microprocessor with power and impedance limits. When the values exceed predetermined power or impedance values, a warning can be given on a user interface and display, and additionally, the delivery of radiofrequency energy can be reduced, modified or interrupted. A control signal from microprocessor can modify the power level supplied by an energy source.

Referring to FIG. 8, in a further embodiment, the VIPER 10 includes an optical fiber 90 to provide target specific viewing to ensure presence of the stylet 20 and tines 80 within the proximity of the desired target tissue area. The optical fiber 90 within the stylet 20 of the apparatus 10 is preferably enclosed by the mandrel 60 which houses one or more optical fibers 90 and one or more tines 80. One or more optical fibers 90 are used to image in the vicinity of the nerve tissue ablation area to first determine that the energy delivery 120 and receptor tines 121 have been deployed and located in the proper tissue type. During operation, the optical fiber 90 with a monitoring and control unit is used to determine the extent of ablation within the targeted area. The monitoring and control unit images 3 to 4 mm ahead of the targeted ablation surface to enable a user to see sensitive tissue and to assess the status of ablation. The optical fibers 90 are operatively connected and multiplexed to an external optical coherence domain reflectometer. The optical coherence domain reflectometer is operatively connected to the system monitoring and control unit. While not shown, the optical fibers 90 are provided at their distal ends with optics, such as gradient index lenses or prisms, to capture images of different areas of the ablation field.

In operation, the light transmitted and received by the optical fiber 90 is processed by a control module to allow continuous imaging of ablation surfaces as the tines 80 are drawn from their furthest extent back to a fully enclosed position within the stylet 20. This incremental imaging feedback can be used in conjunctions with other sensors 150 to determine temperature and impedance, as a further means of confirming adequate ablation of the nerve tissue in the target area.

In a further embodiment, the operation of the system including the VIPER 10 and ultra-wide band monitoring (“UWB”) and imaging are described. An UWB scanning sensor is placed on the subject's skin at a location in close proximity to the target site. The UWB scanning sensor and associated imaging system is then activated to create a two or three-dimensional image of the tissue in close proximity to the target site. Unlike other imaging systems including X-ray fluoroscopy, the UWB scanning system creates a fully resolved image of the target site including the bones in close proximity to the target site. This allows placement of the VIPER 10 to be monitored continuously throughout deployment without relocating the UWB scanning sensor. X-ray fluoroscopy may sometimes require re-positioning of the patient, delaying the procedure and creating additional discomfort for the patient.

In certain circumstances, a dual UWB scanning platform is placed on the patient in close proximity to the target tissue site, providing a three-dimensional image of the target tissue site and surrounding anatomy. Additionally, the UWB scanning platform may be incorporated with an X-Y motion system to provide additional scanning coverage. The physician may then use three-dimensional imaging techniques to monitor and navigate the VIPER 10 to the desired target tissue site in real time.

Once the 2-D or 3-D UWB scanning sensor has been placed, activated and used to create an image of the target tissue site, the physician will then begin to deploy the VIPER 10 percutaneously toward the target site. The UWB scanning platform and the monitoring and control unit continues to monitor and image the area about the target tissue site throughout the procedure.

The monitoring, control and feedback unit of the UWB scanning system will automatically alert the physician if the VIPER 10 is navigated too closely to other sensitive anatomy or tissue, such as another critical nerve fiber. The monitoring, control and feedback unit of the UWB scanning system, in combination with a VIPER three dimensional imaging and navigation system, may also be used to train other physicians in the subject procedures, on either cadavers or dummy systems.

Once the VIPER 10 has been deployed to a target tissue site and the desired energy delivery technique has been activated, the UWB scanning platform will track the development of the desired lesion. As the target tissue is heated, the UWB scanning platform will provide a real-time update of the lesion status by tracking the dielectric constant of the tissue. The dielectric constant of the original tissue will differ from that of the ablated tissue. The original tissue is comprised of cell material and fluids. Once the target site has been ablated, the ablated tissue will have minimal fluids and other cellular material will have been driven off from the target tissue site by the thermal energy. Additional various chemical changes, including protein coagulation of the target tissue, will cause the signal the signal received by the UWB system to change as ablation proceeds.

For clarity, specific methods of use of the apparatus and system according to the invention are described. The methods described are particularly suitable to perform treatment of pain fibers in the spinal structure, but may also be adapted to provide effective ablation of other tissue types or at other anatomical location. The following methods describe application of the apparatus to specific regions of the spine, including the cervical, thoracic, lumbar, and sacroiliac regions. In certain of the described procedures, a VIPER 10 having two tines 80 or three tines 80 may be preferable.

Generally, three tines 80 will be preferred whenever it is desirable to create a lesion of much greater size, or, where three tines 80 are required to envelope an area to maximize the probability of actually ablating targeted tissue.

1) Lumbar Paravertebral Sympathetic Ganglion Nerve Tissue Radiofrequency Method and Procedure

Prior Art Method: The approach used for treating the subject nerve tissue using a traditional single needle approach is discussed. In the spinal lumbar region, the sympathetic ganglion nerve chain lie antero-lateral to the lumbar vertebrae. Traditionally, multiple individual needles are used to adequately ablate the targeted nerve tissue in this procedure. The needles are typically 18, 20 or 22-gauge insulated cannulae, each having a 10 mm curved active tip. The single needles are placed in the patient from a posterior oblique position using fluoroscopy, after aligning the tip of the transverse process of the targeted vertebra with the visible edge of the vertebral body. The single needles are then steered to the antero-lateral position of the vertebral bodies L2, L3 and L4. Although chained, the ganglion bodies are diffuse. Consequently, this single needle approach suffers from limited lesion effect where the critical ganglion bodies may be only partially ablated or missed altogether. Hence, accuracy, reliability and effectiveness of the single needle approach are limited, causing inconsistent and unpredictable patient outcomes. A more reliable method for thoroughly ablating target ganglion tissue is needed to improve patient outcomes.

VIPER Method: The approach used for treating the lumbar paravertebral sympathetic ganglion nerve tissue with the VIPER 10 ablation apparatus is discussed. In this procedure, it is preferable to use VIPER 10 having three tines 80 for targeted nerve tissue radiofrequency ablation.

The VIPER 10 has several design characteristics that facilitate directional control, appropriate placement, and accuracy and broader coverage for more effective ablation results. At the outset of a procedure, the curved VIPER 10 is steered and placed in a fashion similar to that of a curved single needle, but with distinct advantageous features. First, the blunt but sharp nose 29 of the stylet 20 of the VIPER 10, in conjunction with the curved forward portion 28 of the stylet 20, facilitates sliding the VIPER tip 24 along the vertebral bone to the appropriate target location.

A typical sharp tip on a curved needle would tend to impale the vertebral body bone and hence, is difficult to reposition for accuracy. Consequently, the VIPER 10 includes a distal rounded sharp nose 29, thereby avoiding hang-ups during placement and positioning as the VIPER 10 is able to tangentially slide over the boney surface of the vertebra.

After confirmation of initial proper placement of the VIPER 10 in the sympathetic ganglion chain, the VIPER 10 would then be withdrawn several millimeters. Next, the VIPER tines 80 would be advanced from within the stylet 20 of the VIPER 10 toward the target tissue. As the tines 80 extend from the stylet 20, they will diverge from each other in an outward curving, flaring geometry to achieve a variably wide and long lesion coverage.

Position and placement of the VIPER 10 would be confirmed by fluoroscopy assessment, as with single needle procedures. However, with the VIPER 10, confirmation of targeted placement adjacent ganglion tissue would be further confirmed by direct view of actual tissue via an optical fiber 90. Further, targeted placement of the VIPER 10 may also be confirmed using ultra-wide band radiofrequency scanning technology according to the present invention.

Additionally, with reference to FIG. 9, during the course of the procedure, the tines 80 may be rotated as one unit 360 degrees about the center axis of the stylet 20 while compacted within the stylet 20. After rotation and subsequent extension, the tines 80 then contact a separate portion of the target tissue site, improving overall control of lesion positioning and shape. The ability to rotate the tines 80 of the VIPER 10 facilitates more accurate placement and coverage, and, increases the amount of coverage that can be addressed by the VIPER 10. The curve of the tines 80, in conjunction with the flaring of the tines 80, allows variable width and depth of displacement of the tines 80 to maximize the probability of capturing and ablating the desired target tissue.

As illustrated in FIG. 9, the three-dimensional variable placement of the tines 80 substantially increases the coverage area, improving the likelihood that the desired tissue ablation will be achieved. Compared to the prior art approach of using a single needle placed multiple times, the VIPER 10 has the potential to provide substantially more lesioning effect using a single placement step.

As illustrated in FIG. 9, while the stylet 20 of the VIPER remains in one location, the tines 80 may be repeatedly adjusted and reoriented to increase the coverage of the tines 80, creating a larger lesion area. The ultimate benefit from the ability of the VIPER 10 to flare and rotate its tines 80 is improved patient outcomes, and, increased patient comfort. A physician will be able to more effectively treat a patient without causing the patient to feel like a pin-cushion during the procedure. Further, by increasing the probability that the appropriate tissue will be ablated during the initial procedure through use of the VIPER 10, the need for additional painful needle placement is minimized and patient outcomes are greatly improved, as is patient satisfaction with the procedure.

Prior to initiation of a radiofrequency lesion, sensory and motor stimulation assessment of the patient is used to reinforce and confirm proper positioning of the VIPER 10. This is done to avoid lesion damage to somato-sensory nerves that lie in close proximity to the sympathetic ganglia.

Once proper positioning is confirmed, and, also prior to initiation of the radiofrequency lesion, local anesthesia is injected through an infusion port 70 located on the stylet 20 of the VIPER 10.

Depending on the circumstances of the particular patient case, heating protocols will be varied. For non-destructive lesions, tests indicate that appropriate heating ranges will be between 42 to 44 degrees Centigrade for a duration of 2 to 4 minutes. For destructive lesions, the heating range is typically above 44 degrees Centigrade. For example, full ablative destruction normally requires a tissue temperature of 80 degrees with application for a range of between 60 and 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

2) Cervical Facet Radiofrequency Neurolysis

Prior Art Method: The approach used in the prior art for treating the subject nerve tissue via cervical facet radiofrequency neurolysis using a traditional single needle approach is discussed. When a patient presents with symptoms of well-circumscribed pain overlying the cervical facet joints, there is a good possibility that the joints are involved in the problem. Affected patients commonly report pain in the neck and shoulder girdle with associated headaches and even ear pain. Diagnostic injections of the facet joints can assist in making the diagnosis but can be associated with false positive outcomes. The anatomy of the cervical facet joints includes a medial branch which wraps around in a posterior fashion and sends branches along the waist of the cervical facet column at two levels for each medical branch. Consequently, it is normally prudent to perform lesions at multiple levels to properly denervate a specified facet joint. Cervical facet radiofrequency neurolysis is technically challenging. Traditionally, practitioners use individual 18, 20 and 22 gauge needles that are either curved or straight. The needles placed along the waist of the articular pillars of each facet joint at different locations depending upon the level. Multiple adjustments are required to cover a large enough area to have confidence that at least one of the radiofrequency lesions is close enough to a medial branch nerve to effect an ablation. From a posterior to anterior approach, some use a 90 degree placement plus a 30 degree angled approach to be more effective. The best results appear to be with the use of a 16 gauge “Ray” needle which is no longer made. The larger diameter 16 gauge Ray needle produces a wider lesion area compared to the 22 gauge needle. Even so, multiple placements still must be made to insure coverage and the process may take hours to complete. Studies associated with the use of the large Ray needle have shown improved patient outcomes which are generally believed to be caused by the increased lesion size. The VIPER 10 allows a larger lesion size without having to increase the size of the introduced needle.

VIPER Method: The approach used for treating the subject nerve tissue via cervical facet radiofrequency neurolysis with the VIPER 10 is discussed. The VIPER 10 allows substantial increase in lesion size while using a smaller size stylet 20, creating increased patient comfort and significantly shortening the time required for treatment.

According to the present invention, a VIPER 10 using either two tines 80 or three tines 80 would provide the necessary width and curve to insure adequate coverage in a single placement of the VIPER 10. From a posterior, slightly caudad, direction the curved VIPER 10 is steered toward the desired position on the waist of the articular pillar of the cervical facet joint to touch the lamina. The stylet 20 of the VIPER 10 is then withdrawn slightly and the curved tines 80 of the VIPER 10 are advanced to spread out laterally and project anterior-medial to enable a lesion varying from 4 to 6 mm long and 2 to 4 mm wide.

As with other procedures, prior to the radiofrequency lesion process, sensory and motor stimulation is used to reinforce proper positioning of the VIPER 10. Additionally, prior to the radiofrequency lesion process, local anesthesia may be injected through the infusion port 70 of the VIPER 10.

Heating protocols will vary. Current recommended heating ranges are between 42 to 44 degrees Centigrade for 2 to 4 minutes for creation of a non-destructive lesion. Destructive lesions require thermal temperatures above 44 degrees Centigrade. For example, tissue destruction can be expected by maintaining 80 degrees Centigrade for a range of between 60 to 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

3) Dorsal Root Ganglion Radiofrequency Neurolysis

Prior Art Method: The appropriate radiofrequency procedure for the dorsal root ganglia relies on pulsed application of radiofrequency energy. Prior art approaches include either guiding the tip of a single needle to impact near the dorsal root ganglion nerve bundle on the posterior surface, or, guiding the single needle to lay along side the dorsal root ganglion nerve bundle.

VIPER Method: Here, the VIPER 10 having three tines 80 is the preferred apparatus. In a VIPER 10 with three tines 80, the central tine 80 is typically an energy receptor tine 121; the two outside tines 80 are energy delivery tines 120. In addition to approaching the dorsal root ganglion from the posterior surface, the VIPER 10 having three tines 80 has the capability of “wrapping around” the dorsal root ganglia to some extent and may provide an enveloping field to cause partial ablation of a greater areal extent of the dorsal root ganglia. To achieve this result, the VIPER 10 is first guided toward the dorsal root ganglion. The slightly blunt curved tip 29 of the VIPER 10 is easier to guide than a blunt single needle, and, less likely to damage the dorsal root ganglion than a sharp single needle. If desired, placement of the VIPER 10 can be facilitated by injection contrast through the infusion port 70 on the stylet 20 to outline the dorsal root ganglion. Through the assistance of the curved forward portion 28 of the stylet 20, the VIPER 10 can be rotated appropriately to approach the dorsal root ganglion in a centered posterior position so that the tines 80 of the VIPER 10 may be advanced to partially wrap around the dorsal root ganglion. Temperatures and time appropriate for pulsed radiofrequency procedures can then be applied.

Heating protocols will vary. Heating ranges are between 42 to 44 degrees Centigrade for 2 to 4 minutes for creation of a non destructive lesion. Destructive lesions require thermal temperatures above 44 degrees Centigrade, for example 80 degrees Centigrade for a range of between 60 to 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

4) Lumbar Facet Radiofrequency Neurolysis

Prior Art Method: The current ablation approach uses a straight or curved radiofrequency needle, usually 22, 20, or 18 gauge. The needle is first introduced from a slightly caudad oblique entry point following the fluoroscopic beam to lie parallel to the junction of the superior articular process and the transverse process. This entry orientation is designed to bring the needle along side the medial branch of the dorsal ramus nerve for maximum radiofrequency heat exposure to the nerve supply to the facets. Part of the orientation is also intended is to lay the tip of the needle on the anterior junction edge to impact the nerve before it dives below the mamillary ligament.

One of the difficulties associated with the current approach is the difficulty in using fluoroscopy to determine where the junction of the superior process and the transverse process of the lumbar vertebra are located. Another difficulty is that the mamillary ligament may shield the targeted nerve over much of its course from heat penetration.

VIPER Method: Here, the VIPER 10 having three tines 80 is generally preferred. The VIPER 10 is designed to accurately locate the junction of the SAP/TP as well as penetrate the mamillary ligament and still provide a variable heat lesion along the length of the nerve. The VIPER 10 can be placed either from the current parallel approach, as just described, or from a perpendicular approach. If the VIPER 10 is placed parallel, the VIPER 10 can be flared to create a much wider coverage than 22, 20 or 18 gauge standard needles, substantially increasing the probability that the tines 80 will lay over the target nerve. This benefit is expected whether the target nerve is at the anterior edge of the SAP/TP junction or posterior along the groove. The tines 80 of the VIPER 10 have sufficient rigidity and sharpness to penetrate the mamillary ligament.

Alternatively, the VIPER 10 may be placed from slightly cephalo-oblique position to impact along the TP and up against the SAP to insure precise and safe placement. The curved distal forward portion 28 of the VIPER 10 enables guiding the VIPER 10 to impact the TP lateral but proximate to the SAP/TP junction. The slight blunt tip 29 facilitates sliding the VIPER 10 along the TP to impact the SAP in the SAP/TP groove.

The optical fiber 90 facilitates confirmation of placement. Ideal placement will be near the anterior superior border of the SAP/TP. The stylet 20 of the VIPER 10 then is withdrawn a variable length, for example, 3 to 6 mm, and the tines 80 are advanced to impact against and advance up along the SAP enabling both a wide and relatively long lesion area not possible with single needle placement.

As with other radiofrequency ablation procedures described, prior to the radiofrequency lesion process, local anesthesia may be injected through the infusion port 70 in the stylet 20 of the VIPER 10. Heating protocols will vary. However, current heating ranges required are between 42 to 44 degrees Centigrade for 2 to 4 minutes for non-destructive lesions. Destructive lesions require tissue temperatures above 44 degrees Centigrade, for example, 80 degrees Centigrade for a range between 60 and 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

5) Sacroiliac Joint Radiofrequency Neurotomy

Prior Art Method: The approach used in the prior art for treating the subject nerve tissue via sacroiliac radiofrequency neurolysis using a traditional single needle approach is discussed. Radiofrequency applied to sacroiliac-mediated pain is undergoing vigorous research. It is generally believed that most of the nerve supply to the joints emerges from the sacral nerves and traverses laterally to the joints as well from an inferior coursing nerve from L5. The sacroiliac joint can produce symptoms quite similar to facet joint abnormalities. Pain emanating from the sacroiliac joint may cause buttock pain and referred mechanical symptoms. Areas of referral included the hip, groin, anterior thigh and calf. Most current prior art approaches to radiofrequency ablation of nerve fiber in this region rely on the use of multiple single needle placements to intersect the nerves in a perpendicular manner lateral to the posterior neural foramen of S1, S2, S3 and S4 and from the sacral ala position towards the S1 joint. The intent of this method is to use the long, 10 mm active tip to create and place a long linear lesion in front of the neural foramen and sacra ala. Unfortunately, creating a number of connecting linear lesions using this method creates an extremely long procedure which can prove to be very uncomfortable for the patient, and, can tend to expose the treating physician to high doses of X-ray radiation emitted by the fluoroscopy system.

Another approach is to ablate along the inner ridge of the posterior neural foramen from the 20 o'clock to 6'clock position laterally.

Another approach is to place multiple needles along the posterior sacroiliac joint in the ligaments and potentially in the joint in order to apply bipolar radiofrequency ablation.

VIPER Method: The approach used for treating the subject nerve tissue via sacroiliac joint radiofrequency neurotomy with the VIPER 10 is discussed. Here, the VIPER 10 having either two tines 80 or three tines 80 may be used interchangeably. The VIPER 10 can be placed in a fashion similar to that described above for the prior art approach. However, the VIPER 10 is much more desirable due to its ability to lay down a much wider lesion than that produced by a single needle approach.

Alternatively, the VIPER 10 can be introduced either from lateral-to-medial or medial-to-lateral with fluoroscopic assistance to be guided just lateral to the foramen. The curved portion of the VIPER 10 facilitates steering to a point of impact and then, the blunt tip 29 facilitates sliding the stylet 20 of the VIPER 10 to the appropriate desired location. Verification of placement may be facilitated by the optical imaging capabilities of the VIPER 10. The tines 80 of the VIPER 10 can be deployed to flare in front of the foramen from the lateral 2 o'clock to 6 o'clock position to lie over the target nerves, thus obviating the need to place the VIPER 10 in the neural foramen. Multiple placements of the tines 80 of the VIPER 10 without relocating the stylet 20 can more easily increase the lesion path to provide an improved outcome with minimal discomfort to a patient.

Further, in another alternative approach, the VIPER 10 can be deployed into the ligaments and joint at the posterior joint interface for more efficient bipolar lesioning.

Prior to the radiofrequency lesion, sensory and motor stimulation is used to reinforce proper positioning. Prior to the radiofrequency lesion, local anesthesia may also be injected through the infusion port 70. For non-destructive lesions, heating ranges between 42 to 44 degrees Centigrade for 2 or 4 minutes is required. Destructive lesions require tissue temperatures above 44 degrees Centigrade, for example, 80 degrees Centigrade for a range of between 60 to 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

6) Splanchnic Nerve Radiofrequency Ablation

Prior Art Method: The approach used in the prior art for treating the subject nerve tissue via splanchnic radiofrequency ablation using a traditional single needle approach is discussed. The standard, prior art approach to chemical neurolysis of the celiac plexus is to pass a 22 gauge needle through the aorta to the celiac plexus. A safer alternative to a chemical neurolysis of the celiac plexus is a radiofrequency lesion or ablation of the splanchnic nerve. This nerve courses along the anterior-lateral border of the T10 and T11 levels. Usually, radiofrequency needles are laid along the antero-lateral border of the T11 vertebrae with two or more re-positionings to provide extended coverage of the radiofrequency lesion.

VIPER Method: The approach used for treating the subject nerve tissue via splanchnic nerve radiofrequency ablation with the VIPER 10 is discussed. The VIPER having two tines 80 or three tines 80 may be used interchangeably for this application. A single VIPER 10 is laid along the T11 vertebrae in the designed navigable fashion. When the position is appropriate, the stylet 20 would be slightly withdrawn and then the tines 80 would be deployed. Position would be confirmed with UWB, optical visualization, fluoroscopy, or stimulation before the lesion is made.

Prior to the radiofrequency lesion, sensory and motor stimulation is used to reinforce proper positioning. Prior to the radiofrequency lesion local anesthesia may be injected through the infusion port 70 on the stylet 20. Non-destructive lesions require heating ranges between 42 to 44 degrees Centigrade for 2 or 4 minutes. Destructive lesions require tissue temperatures above 44 degrees Centigrade, for example, 80 degrees Centigrade for a range of between 60 to 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

7) Stellate Ganglion Radiofrequency Procedure

Prior Art Method: The Stellate Ganglion is a diffuse structure that is a combination of the inferior cervical ganglion and the first thoracic ganglion. It is located at the base of the C7 transverse process and extends inferior to T1, along the anterior lateral border of the C7 and T1 vertebral bodies.

With the assistance of fluoroscopic guidance, the prior art approach is to place a single 22 gauge needle with a 4 to 5 mm active tip from anterior to posterior down to the base of the transverse process at C7. If the needle is too medial it will contact the vertebral body and not be in contact with the ganglion body. Oblique views help determine that the needle is lateral enough. Contrast injected will help to confirm placement. 50 Hertz stimulation is accomplished before RF treatment. Historic high temperature destructive lesions have given way to non-destructive pulsed RF to increase patient comfort and safety.

VIPER Method: According to the present invention, the VIPER 10 having three tines 80 is generally recommended for stellate ganglion radiofrequency procedures, but configurations with either two or three tines 80 may be used interchangeably. Either will provide a wider coverage than the single 22, 20 or 18 gauge needles currently available.

The VIPER 10 would be advanced to contact the anterior lateral border of the vertebral body medial to the transverse process at C7. The stylet 20 would then be slightly retracted and the tines 80 then would be advanced from the stylet 20 in a flared fashion to contact the base of the transverse process at C7. Position would be confirmed by fluoroscopy as well as directly with fiber optics. Stimulation and RF treatment would be carried out in the usual manner.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

8) Thoracic Facet Radiofrequency Procedure

Prior Art Method: The medial branch nerve supply to the thoracic facets is very variable. Currently, it is generally believed that the nerves course over the outer third of the transverse process, in a superior position.

The current technique in use is similar to the approach for lumbar facets. A single needle is placed from a caudad oblique position to be steered down to the transverse process to an assumed appropriate position. Multiple lesions must be made in order to increase the probability that the nerves will be ablated.

Because of the inherent inaccuracy of this approach, others advocate bringing a single needle in from a lateral approach to intersect the nerves in a perpendicular fashion. This is intended to create a 10 mm long lesion that lies over the nerves.

VIPER Method: The VIPER 10 having three tines 80 is generally recommended for the thoracic facet procedure. The VIPER 10 by design will be able to provide substantially greater lesion coverage, whether the approach is parallel or perpendicular to the nerves.

Irrespective of the approach mode, the stylet 20 with the tines 80 in a withdrawn compacted configuration would be steered down to impact on the superior-lateral third of the transverse process of the thoracic vertebra above T11. T11 and T12 would be approached as if lumbar. The tines 80 would then be deployed from the stylet 20 to a length of about 10 mm, thus providing a variable width lesion of about 4 to 6 mm. For improved outcomes, the tines 80 can be rotationally repositioned for a second lesion to cover part of the medial third of the transverse process.

Prior to the radiofrequency lesion process, local anesthesia may be injected through the infusion port 70 in the stylet 20 of the VIPER 10. For non-destructive lesions, current heating ranges require between 42 to 44 degrees Centigrade for 2 or 4 minutes. Destructive lesions require tissue temperatures above 44 degrees Centigrade, for example, 80 degrees Centigrade for a range of between 60 to 90 seconds.

The VIPER's fiber optic imaging capability provides an additional capability beyond existing solutions to tangibly identify the tissue type in proximity to the tines 80 of the VIPER 10. Additionally, through spectral analysis, the optical fibers 90 will provide additional thermal assessment and feedback for appropriate monitored control of the lesion. This data can be used to supplement and verify data from sensors 150, such as temperature and impedance, minimizing the need for the physician to rely on approximations for intensity and duration of application during the ablation process.

The foregoing descriptions of preferred embodiments of the invention have been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A spinal nerve tissue ablation apparatus, comprising:
a stylet including a first electrode tine and a second electrode tine, each electrode tine having a tissue piercing distal end and positionable in the stylet as the stylet is advanced through tissue, the first and second electrode tines being deployable with curvature from the stylet; and
a linear control member coupled to at least one of the first or the second electrode tines, the linear control member adapted to advance the first and second electrode tines into and about a nerve fiber, the linear control member including an actuable portion having a diameter or shape configured to control or limit movement of the linear advancement member, at least a portion of the linear control member being positionable in the stylet.

2. The apparatus of claim 1, further comprising:
a sensor tine with a tissue piercing distal end, the sensor tine positionable in the stylet as the stylet is advanced through tissue, the sensor tine being deployable from the stylet with curvature equivalent to that of the first and second electrodes tines.

3. The apparatus of claim 2, wherein at least a portion of the sensor tine is an electrode.

4. The apparatus of claim 2, further comprising a sensor coupled to a distal portion of the sensor tine.

5. The apparatus of claim 1, further comprising:
a rotational means for rotating the linear control member within the handle causing the electrode tines to likewise rotate within the stylet.

6. The apparatus of claim 5 wherein the electrode tines and sensor tines have similar outward grasping curvature such that rotation by the rotational means causes the distal ends of the electrode tines and sensor tines to reach other portions of a targeted tissue site in a multivariable cylindrical coverage manner.

7. The apparatus of claim 1, wherein at least a portion of the electrode tine has a tissue piercing distal end that is ultrasonically viewable.

8. The apparatus of claim 1, further comprising an inner cylindrical mandrel.

9. The apparatus of claim 8, wherein the mandrel may slidably reciprocate within the stylet.

10. The apparatus of claim 8, wherein the mandrel includes two or more lumens for slidably receiving tines.

13. The apparatus of claim 8, wherein the mandrel includes an insulation that electrically isolates the tines from the mandrel.

15. The apparatus of claim 1, further comprising:
a first sensor coupled to the first electrode tine and a second sensor coupled to the second electrode tine.

16. A spinal nerve tissue ablation apparatus, comprising:
a stylet with a distal end sufficiently sharp to penetrate tissue; and
including a first electrode tine, a second electrode tine and a third electrode tine, each of the first, second and third electrodes having a tissue piercing distal end positionable in the stylet as the stylet is advanced through tissue, the first, second and third electrodes being selectably deployable with equivalent curvature from the stylet to a tissue site; and
a mandrel positionable within the stylet and coupled to at least one of the first, second or third electrodes, the advancement member adapted to advance the first, second and third electrodes into and about a nerve fiber tissue and be substantially parallel with respect to a longitudinal axis of the stylet.

17. A spinal nerve tissue ablation apparatus, comprising:
a stylet with a distal end sufficiently sharp to penetrate tissue;
a first electrode tine and a second electrode tine each having a tissue piercing distal end and positionable in the stylet as the stylet is advanced through tissue, the first and second electrode tines being selectably deployable with curvature from the stylet to a tissue site;
a sensing tine, the sensing tine having a tissue piercing distal end and positionable in the stylet as the stylet is advanced through tissue, the sensing tine being deployable from the stylet with curvature equivalent to that of the first and second electrode tines;
a mandrel coupled to at least one of the first or the second electrode tines or the sensing tine, the mandrel adapted to advance the first or second electrode or the sensing tine into a nerve tissue fiber, the mandrel including an actuable portion having a diameter or shape configured to control or limit movement of the mandrel, at least a portion of the mandrel being positionable in the stylet; and
a sensor coupled to the sensor tine.

18. The apparatus of claim 17, wherein the device operates in a mono-polar manner.

19. The apparatus of claim 17, wherein the device operates in a bi-polar manner.

20. A spinal nerve tissue ablation apparatus, comprising:
a stylet with a distal end sufficiently sharp to penetrate tissue; and including a first electrode tine and a second electrode tine, each electrode having a tissue piercing distal end and positionable in the stylet as the stylet is advanced through tissue, the first and second electrode tines being deployable with curvature from the stylet; and
a mandrel positionable within the stylet and coupled to at least one of the first or the second electrode tines, the mandrel adapted to advance the first and second set of electrode tines into and about a nerve fiber, during advancement, the advancement member configured to be substantially parallel along its entire length with respect to a longitudinal axis of the stylet.
